(19) United States
(12) Patent Application Publication
(10) Pub. No.: US 2004/0006259 A1
(43) Pub. Date: Jan. 8, 2004

(54) Method and device for determining migration pathways of cells

(76) Inventors: Christoph Pedain, Munchen (DE); Andreas Hartlep, Munchen (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 10/464,809

(22) Filed: Jun. 18, 2003

Related U.S. Application Data
(60) Provisional application No. 60/437,563, filed on Dec. 31, 2002.

(30) Foreign Application Priority Data
Jun. 19, 2002 (EP) 02 013 611.5

(51) Int. Cl. A61B 5/00

(52) U.S. Cl. 600/300

(57) ABSTRACT

A method for determining migration pathways of cells includes capturing data on a body using an imaging method. Information relevant to migration pathways is obtained from the captured data and migration pathways are individually calculated for said body. A device for determining migration pathways of cells includes a device for carrying out an imaging method and an evaluation unit for individually ascertaining migration pathways in a body, based on the data provided by the device.

### RELATED APPLICATION DATA

This application claims priority of U.S. Provisional Application No. 60/437,563, filed on Dec. 31, 2002, which is incorporated herein by reference in its entirety.

### FIELD OF THE INVENTION

The present invention relates to a method and a device for determining or modeling migration pathways of cells, such as tumor cells, in tissue.

### BACKGROUND OF THE INVENTION

Analytical in-vivo methods are known from U.S. Pat. No. 6,284,219 B1, which enable diseases involving discrete biochemical transmission pathways to be diagnosed. In this way, a particular enzyme is administered and a particular product of the enzyme's effect on a body is measured, where the incidence and concentration of the product can be related to a disease to be examined. The rate of enzyme-to-product conversion is determined, and an analysis is carried out on this basis. Thus, the intention is to improve how the treatment is carried out by determining a metabolic function in-vivo.

Known biological and chemical methods can provide information about diseases already present, but it is not possible using these methods to individually determine or predict the migration pathways of cells.

It is known that tumors, such as glioplastomae, are very likely to cause metastasis. An individual tumor cell or a small group of cells separates, for example, from an existing tumor and migrates into the surrounding tissue or blood stream. From these cells, new tumors can develop at other locations, even if the primary tumor has been treated, for example, by resection or cutting it out, radiotherapy, local chemotherapy or other treatments. Tumor cell migration is patient-specific and differs from individual to individual due to different anatomical circumstances, such as the tumors forming differently, etc., and cannot be directly and precisely predicted by known methods. However, knowledge of the migration pathways of these cells would enable metastasis, which may develop, or small metastasises, which have already developed, which using known methods can often only be detected beyond a certain size, to be identified and treated early. Since in the early stages, metastasis can be treated relatively easily and with a high probability of success. Since treating small metastasises generally has no significant side effects, new and particularly promising treatments may be carried out by knowing and predicting the migration pathways of cells as exactly as possible.

### SUMMARY AND DISCLOSURE OF THE INVENTION

In accordance with one aspect of the invention, a method and a device for determining migration pathways of cells, in which migration pathways can be determined or predicted individually, are provided.

In one embodiment of the method for determining migration pathways of cells, such as, for example, tumor cells or cells introduced into a body or organism by infusion or injection, specific or anatomical body data, which is individually characteristic of a body, can be captured and relevant for ascertaining migration pathways. The specific or anatomical body data can include, for example, data relating to the shape of a tumor, the structure and type of a tissue, data relating to the course of the blood stream, the shape and location of hollow spaces, cysts, oedemas or other data characteristic of a body, a body structure or tissues.

The data is preferably captured using imaging methods, such as, for example, nuclear magnetic resonance (MRI) methods, ultrasound methods, computer tomography (CT) methods, X-ray, SPECT (Single Photon Emission Computed Tomography) methods such as for example PET, or other methods. Other or further examination or measuring methods can also optionally be used to obtain data on the body, such as the body structure properties or the tissue properties. Such data can be obtained, for example, from a biopsy. From the data obtained in this way, specific to a body, for example a person, specific anatomical information can be ascertained in order to determine migration pathways of cells starting from a particular area or point, for example, a tumor. For this purpose, mathematical models or findings obtained from other examinations, as described, for example, in U.S. Pat. No. 6,284,219 B1, which is incorporated by reference herein in its entirety, can be adduced.

For example, it is possible to examine in general how a particular type of cell moves in a particular type of tissue or in a blood stream. From the specific anatomical information obtained in this way, the specific migration pathways possible for a particular tumor can be calculated. Probabilities can also be given for the fact that cells from a particular tumor or area of tissue spread over as specific migration pathway of the migration pathways ascertained in this way and can threaten other areas of the body or tissue, such that suitable countermeasures for preventing migration and/or preventively treating possible metastasis can be introduced at an early stage.

In general, non-imaging methods can also be used for obtaining body data, if possible migration pathways can be calculated using these data. It is likewise possible, in accordance with the invention, to ascertain not only migration pathways of cells, but also migration pathways of other substances in a body or tissue.

In accordance with the invention, data can also be obtained from measurements relating to the transport mechanisms in a body or tissue. Such known methods are often also referred to as diffusion imaging. Furthermore, it is also possible to use known methods for perfusion imaging to obtain information on how particular substances or cells are transported in a blood vessel or a vascular system. Using methods for perfusion imaging, data on transporting substances or cells at the blood-brain barrier, which is often important for predicting migration pathways, can be obtained in order to examine possible migration pathways for substances or cells. This can be useful, for example, when examining and treating brain tumors, and in order to have additional information available on how and where particular substances or drugs should be used, i.e., injected and/or dosed, in order, for example, to treat a brain tumor.

Combinations of measurements mentioned above, or also individual measurements, can be used, for example, to determine the permeability of tissue in the different spatial directions or to distinguish between anatomical features, such white matter and grey matter.

The individual anatomical information and data obtained in this way can be used both to ascertain or calculate migration pathways of cells. Furthermore, it is also possible to also use this data, and optionally other data relating, for example, to perfusion or diffusion, for planning, preparing and/or executing treatment of a particular area of the body at risk. Data for planning, preparing or executing radio-surgical operations can be ascertained. For example, the area of a tissue to be irradiated and/or the radiation dosage, which is to act on a location, can be ascertained, at which location small metastasis may have formed or may be formed, which cannot yet be located by methods known to date.

Furthermore, it is possible to use the information obtained in accordance with the invention with respect to possible migration pathways and/or areas of the body at risk for planning, preparing and/or executing chemotherapy. For example, points can be determined at which an infusion or injection is to be carried out, wherein information on the type and/or concentration of an agent to be used, such as, for example, a chemotherapeutic agent, can be ascertained. It is to be appreciated that the method in accordance with the invention and the device in accordance with the invention described below can be used in conjunction with a device and method for administering a substance, such as is disclosed in commonly owned European patent application number 01 128 614.3, filed on Nov. 30, 2001, which is incorporated herein by reference in its entirety, and commonly owned U.S. patent application Ser. No. 10/075,108, which is incorporated herein by reference in its entirety. The migration pathways ascertained for a body can likewise be used for determining insertion points of cells, such as, for example, neuronal stem cells or modified cells, which can serve to prevent or treat a possible disease in one or more areas at risk.

Furthermore, it is possible to obtain data for planning, preparing and/or assisting one or more surgical operations by using the possible migration pathways of cells ascertained in accordance with the invention and/or the ascertained areas of the body at risk. Ascertaining the possible migration pathways of cells and/or the areas of the body at risk can make it possible to operate as early as possible on areas of the body or tissue possibly affected by a disease. Known navigation methods for targeted surgical operations can for example be used.

In one embodiment, the information obtained in accordance with the invention on possible migration pathways of cells or substances or on possible areas of a body or patient at risk can be used to locally treat precisely defined areas. This is useful because highly toxic drugs can be used very effectively in small areas, in so-called local chemotherapy, for example, using the device described in the European patent application No. 01 128 614.3, which is incorporated herein by reference in its entirety, and commonly owned U.S. patent application Ser. No. 10/075,108, which is incorporated herein by reference in its entirety. A highly effective, relatively high dosage of toxic substances, which is possible in local areas, cannot be used in known, non-specific chemotherapy, which is not limited to a local area.

Likewise, in a treatment using radiation, relatively high radiation doses can be used only if a relatively small area of tissue is irradiated, for which it is known or assumed that diseased tissue, such as metastasis, can form or have already formed there. By contrast, non-specifically, preventively irradiating large areas of tissue with a high radiation dose is ruled out due to the damage it would do to healthy tissue, which would bear no relation to any possible successful treatment. In accordance with the invention, precisely specified and often relatively small areas of tissue can be treated at an early stage using the knowledge of possible migration pathways of tumor cells, such that the teaching in accordance with the invention enables the growth of a tumor to be predicted and tracked, and suitable countermeasures to be taken as early as possible.

In one embodiment, the method for determining migration pathways of cells, can be carried out iteratively. For example, possible migration pathways of cells or areas of the body at risk can be ascertained in a first step, starting from one or more points or areas in the tissue. Verification methods can be subsequently or additionally carried out in which, for example, test substances are introduced into a body or tissue at particular points. The spreading characteristics of these substances, such as the migration of these substances and their presence at particular predetermined areas, is measured in order to more precisely define, on this basis, the migration pathways determined in accordance with the invention. Furthermore, examinations can be carried out, targeted, for example, at the areas at risk ascertained in accordance with the invention, in order to be able to identify, for example, metastasis, even in the early stages.

The invention further relates to a computer program, which can be loaded into the memory of a computer and comprises sections of software code using which individual steps or a number of steps of the method described above can be performed when the program is run on a computer. The invention further relates to a computer program product stored on a computer-compatible medium or data carrier and comprising the computer program mentioned above.

In accordance with another aspect of the invention, a device for determining migration pathways of cells, such as tumor cells, includes a device for capturing specific data relating to the body or the tissue of a person to be examined. The device for capturing specific data can include a device for carrying out an imaging method. The device for determining migration pathways of cells can further include an evaluation unit, such as a computer system, provided in order to determine migration pathways.

The computer system can be designed in such a way that one or more of the method steps described above can be carried out fully automatically and/or interactively. In this way, conventional computers and/or neuronal networks and/or fuzzy logic can be used to ascertain possible migration pathways of cells in a body or tissue from the measured data. Probabilities can also ascertained for the fact that cells spread over a particular one migration path of the migration paths ascertained, so forming, for example, metastasis in areas at risk. In accordance with the invention, it may, for example, be ascertained at which location or in which area of tissue metastasises arise with a particular probability, such that suitable countermeasures, such as introducing substances or cells, irradiating or surgically operating, can be introduced either preventively or as a treatment. In this way, it is also possible to propose individual measures for a person and to automatically ascertain and output corresponding parameters for these measures, such as, for example, the location of an area to be irradiated, the radiation dose, an infusion or injection point, the dose of a substance to be introduced, or other parameters.

In one embodiment, the device in accordance with the invention can also be used to assist in diagnostic, therapeutic or surgical methods relating to the areas at risk.

### BRIEF DESCRIPTION OF THE DRAWINGS

The invention will now be described by way of preferred embodiments.

FIG. 1 is a flow chart of a method for determining migration pathways of cells in accordance with the invention;

FIG. 2 a flow chart of a method for determining migration pathways and use of the determination information in accordance with one embodiment of the invention; and

FIG. 3 is a schematic illustration of a device in accordance with the invention.

### DETAILED DESCRIPTION OF THE INVENTION

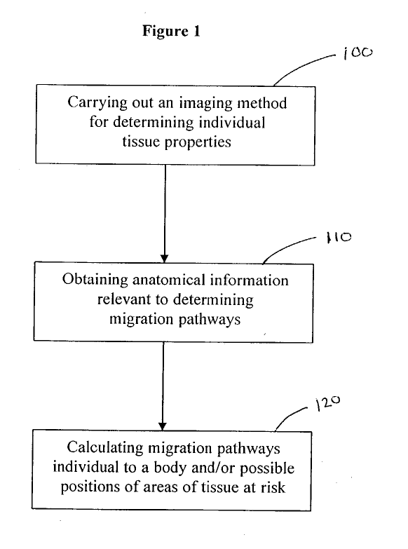

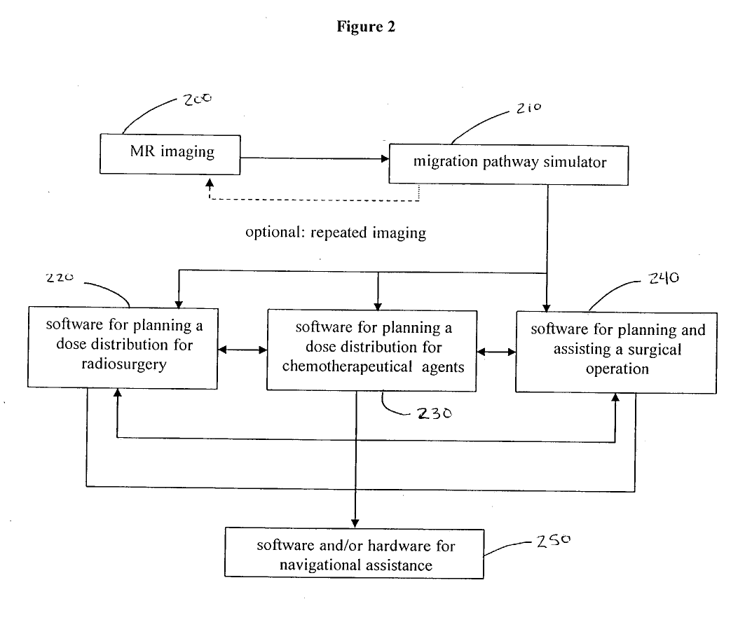

With reference to FIG. 1, a method for determining migration pathways of tumor cells is provided. Body-specific or person-specific tissue properties can be ascertained in a first step 100 using imaging methods. Known devices and methods can be used here, which can capture the arrangement of tissue and of body structures. From the determined tissue properties 100, anatomical information relevant for determining migration pathways of cells is obtained 110. The anatomical information can include the type of tissue, the course of the blood stream or diffusion and perfusion parameters. Anatomical information relevant for determining migration pathways can be combined in order to ascertain the migration pathway of cells 120. The migration pathway of cells can be ascertained beginning from one or more reference points through a tissue having known migration properties and from blood vessels adjacent to the tissue having known perfusion properties, so as to ascertain one particular, a number of, or all possible migration pathways of cells from a particular area into various other areas along various trajectories of a body or tissue. In this way, various migration pathways and possible or probable positions of tumor cells or metastasis in areas at risk can ultimately be ascertained, enabling early and individually tailored treatment of the metastasis in the early stages, as described more fully below with reference to FIG. 2.

With reference now to FIG. 2, an exemplary supplement to the method shown and described in FIG. 1. The information obtained from an imaging method 200, such as for example magnetic resonance imaging (MRI), is inputted into a simulator 210 (also referred to as an evaluation unit) for ascertaining possible migration pathways. Based on the data ascertained by the migration pathway simulator 210, other data can optionally be captured using an imaging method, in order, for example, to verify whether metastasises, which may possibly already be identifiable, are present at the positions or areas of tissue ascertained by the migration pathway simulator 210. Based on the initial data obtained in this way, various types of treatment can be selected, individually or in combination. The type of treatment to be carried out and the parameters specific to the treatment can preferably be generated automatically, for example, by software.

A first possible treatment includes planning and preparing a radio-surgical method 220 in which a radiation device can be triggered, using known navigation methods, in such a way that the metastasises ascertained are preferably irradiated with an optimum dose distribution ascertained by software, given narrowly delimited, defined areas of tissue.

Another possible treatment includes planning a dose distribution for chemotherapeutic agents 230, in which, for example, one or more advantageous locations for introducing chemotherapeutic agents, and the corresponding dose, can be determined, in order to treat the metastasises located in accordance with the invention.

Furthermore, it is possible to provide supplementary information, for example for surgical operations 240. These may include, for example, navigational data 250, using which a surgical operation 240 can be guided to an area at risk including metastasises.

The optional methods of treatment described above can be carried out individually or in combination. The most advantageous possible type of treatment in each case can be selected, optionally, in combination with one or more other methods of treatment. This can be effective, for example, to treat areas of tissue in which tissue at risk or diseased tissue, such as metastasises, ascertained by the method in accordance with the invention or the device in accordance with the invention, is possibly or probably situated.

Measures can also be taken to interrupt identified possible migration pathways or to reduce or restrict the spread of cells. For this purpose, surgical or radiosurgical operations or a treatment using appropriate drugs, such as, for example, chemotherapeutic agents, can be preventively carried out without departing from the scope of the present invention.

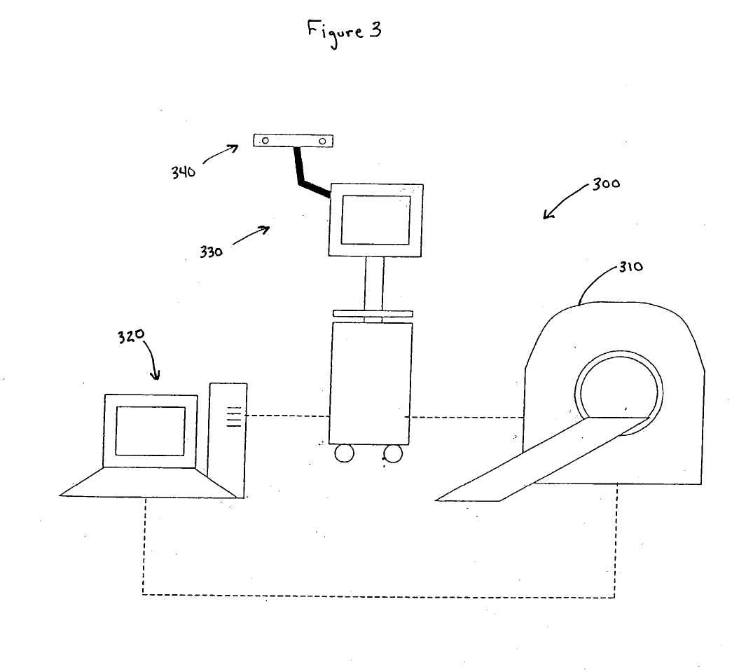

With reference to FIG. 3, a device 300 for determining migration pathways is provided. In one embodiment, the device 300 can include a nuclear spin resonance system 310, such as the VectorVision® system, with which tissue properties of a body can be determined. The determined data can be transferred to a simulation system 320 in order to extract anatomical information relevant for determining migration pathways from this data. The data on possible migration pathways of cells, ascertained by the simulation system 320, can be used to trigger a navigation system 330, so as to be able to carry out a navigated surgical operation, for example using the IR cameras 340, in an area of tissue in which, on the basis of the migration pathways ascertained in accordance with the invention, metastasises are probably or possibly forming. Such navigation systems include those described in commonly assigned U.S. Pat. No. 6,351,659, which is incorporated herein by reference in its entirety.

Although particular embodiments of the invention have been described in detail, it is understood that the invention is not limited correspondingly in scope, but includes all changes, modifications and equivalents coming within the spirit and terms of the claims appended hereto.

What is claimed is:

1. A method for determining migration pathways of cells, said method comprising:
capturing data indicative of tissue properties and body structure properties;
from the captured data, determining anatomical information, said anatomical information including at least one of (i) tissue type, (ii) course of blood stream, (iii) diffusion parameters, and (iv) perfusion parameters; and
calculating at least one migration pathway based on the determined anatomical information.

2. The method as set forth in claim 1, wherein the captured data includes data indicative of at least one of (i) tumor shape, (ii) shape and location of hollow spaces, (iii) cysts, (iv) oedemas, and (v) tissue structure.

3. The method as set forth in claim 1, wherein the data is captured using at least one of (i) a nuclear spin resonance device, (ii) an ultrasound device, (iii) a computer tomography device, (iv) an X-ray device, (v) a SPECT device, and (vi) a PET device.

4. The method as set forth in claim 1, further comprising:
determining a location of at least one area of risk using the individually calculated migration pathways.

5. The method as set forth in claim 1, wherein at least one of (i) diffusion data and (ii) perfusion data are used to determine migration pathways in the body.

6. The method as set forth in claim 1, wherein the migration pathways determined for a body are used to determine at least one insertion point of cells into the body.

7. The method as set forth in claim 4, wherein at least one of (i) the determined migration pathways and (ii) the determined location of areas of tissue at risk are used to set irradiation parameters.

8. The method as set forth in claim 4, wherein at least one of (i) the determined migration pathways and (ii) the determined location of areas of tissue at risk are used to plan and prepare chemotherapeutic treatments.

9. The method as set forth in claim 4, wherein at least one of (i) the determined migration pathways and (ii) the determined location of areas of tissue at risk are used to plan and prepare surgical operations.

10. The method as set forth in claim 4, wherein the imaging method is carried out using at least one of (i) the determined migration pathways and (ii) the determined location of areas of tissue at risk.

11. A computer program which can be loaded into the memory of a computer and comprises sections of software code using which the steps in accordance with claim 1 can be performed when the program is run on a computer.

12. A computer program product stored on a computer-compatible medium or data carrier and comprising the computer program as set forth in claim 11.

13. A device for determining migration pathways of cells in a body, said device comprising:
an imaging device for examining the body; and
an evaluation unit for ascertaining migration pathways in the body based on data provided by the imaging device.

14. The device as set forth in claim 13, further comprising a computational unit for individually calculating migration pathways for the body.
